(19) United States
(12) Plant Patent Application Publication
(10) Pub. No.: US 2012/0110711 P1
(43) Pub. Date: May 3, 2012

(54) Poinsettia plant named 'NPCW10184'

(50) ;

(75) Inventors: Nils Klemm, Stuttgart (DE); Guido von Tubeuf, Stuttgart (DE)

(73) Assignee: KLEMM+SOHN GMBH & CO. KG, Stuttgart (DE)

(21) Appl. No.: 12/925,793

(22) Filed: Oct. 29, 2010

(51) Int. Cl. A01H 5/00

(52) U.S. Cl. PLT/306

(57) ABSTRACT

A new poinsettia plant named ‘NPCW10184’ particularly distinguished by its upright growth habit and intensive marbling color pattern is disclosed.

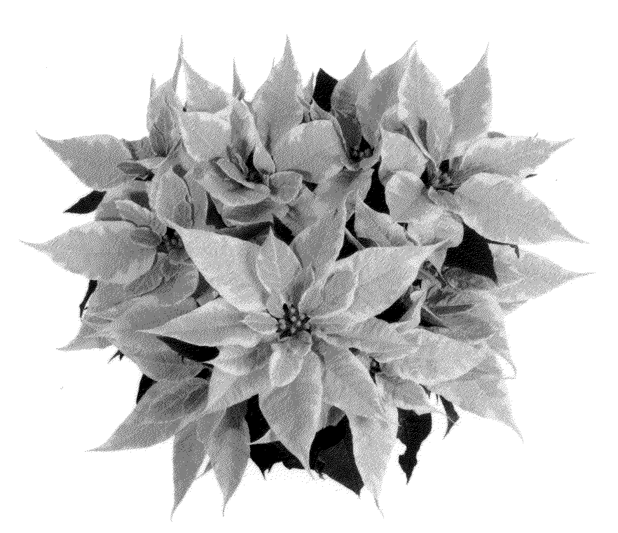

### GENUS AND SPECIES

Euphorbia pulcherrima Willd. ex Klotzsch

### VARIETY DENOMINATION

‘NPCW10184’

### BACKGROUND OF THE NEW PLANT

The present invention comprises a new and distinct variety of poinsettia plant, botanically known as Euphorbia pulcherrima Willd. ex Klotzsch and hereinafter referred to by the variety name ‘NPCW10184’. The new variety is the result of a 30 gray radiation induced mutation in the parent plant ‘Christmas Angel’ (U.S. Plant Pat. No. 19,020) in 2007. A single plant selection was subsequently chosen for further evaluation and asexual propagation.

‘NPCW10184’ was first propagated via vegetative cuttings and has been asexually reproduced by vegetative cuttings in Stuttgart, Germany for over six generations. ‘NPCW10184’ has been found to retain its distinctive characteristics through successive asexual propagations via vegetative cuttings.

‘NPCW10184’ has not been sold or made publicly available more than one year prior to the filing date of this application.

### SUMMARY OF THE INVENTION

The following are the most outstanding and distinguishing characteristics of this new variety when grown under normal horticultural practices in Stuttgart, Germany.
1. Very upright habit; and
2. Intensive marble color pattern.

### DESCRIPTION OF THE PHOTOGRAPH

This new poinsettia plant is illustrated by the accompanying photograph which shows overall plant habit including blooms, and foliage of the plant; the colors shown are as true as can be reasonably obtained by conventional photographic procedures. The photograph is of a whole plant about four-months-old and in full flower.

### DESCRIPTION OF THE NEW VARIETY

The following detailed description sets forth the distinctive characteristics of ‘NPCW10184’. The data which define these characteristics were collected from asexual reproductions carried out in Stuttgart, Germany. The plant history was taken on four month-old plants grown in 13 cm pots from August to December 2009 with a minimum pinch date of 32 weeks. The plants were grown in a greenhouse covered with glass. Color readings were taken under natural light in the greenhouse. Color references are primarily to the R.H.S. Colour Chart of The Royal Horticultural Society of London (R.H.S.) (5^{th} edition 2007).

### DETAILED BOTANICAL DESCRIPTION OF THE NEW PLANT

Classification:
   Family.—Euphorbiaceae.
   Botanical.—E. pulcherrima Willd. ex Klotzsch.
   Common.—Poinsettia.
Parentage: ‘Christmas Angel’ (U.S. Plant Pat. No. 19,020)
Growth:
   Time to produce a rooted cutting.—3 to 4 weeks.
   Blooming habit.—Intermittent; blooms from end of November to January.
   Flowering response time.—Approximately 55 days from beginning of short days until flowering.
   Keeping quality.—Very good.
Plant:
   Form.—Triangular.
   Growth habit.—Upright.
   Height.—20.0 cm to 25.0 cm.
   Width.—20.0 cm to 25.0 cm.
   Number of branches.—5 to 6.
   Average number of inflorescences per plant.—5 to 6.
Stems:
   Color.—Lower part: RHS 135C Upper part: RHS 142B.
   Length.—About 20.0 cm.
   Internode length.—1.0 cm to 3.0 cm.
   Diameter.—0.5 cm to 1.0 cm.
Leaves:
   Quantity.—6 to 10 per lateral branch.
   Arrangement.—Alternate.
   Shape.—Ovate to lanceolate.
   Apex.—Acuminate.
   Base.—Truncate.
   Margin.—Serrate.
   Lobes.—Very few Lobation characteristics: Shallow.
   Texture (both surfaces).—Smooth.
   Vein color (both surfaces).—RHS 135D.
   Variegation.—Absent.
   Size.—Length: 6.0 cm to 12.0 cm Width: 4.0 to 7.0 cm.
   Color.—Mature foliage: Upper surface: RHS 136A Lower surface: RHS 138B Immature foliage: Upper surface: RHS 143B Lower surface: RHS 143C.
   Leaf petiole.—Length: 2.0 cm to 4.0 cm Diameter: 0.2 cm to 0.3 cm Color (both surfaces): RHS 143C.
Inflorescence:
   Lastingness of inflorescence on the plant.—3 to 4 weeks.
   Diameter.—10.0 cm to 16.0 cm.
   Height.—2.0 to 3.0 cm.
Bracts:
   Number of bracts per inflorescence.—8 (with 4 to 6 secondary bracts).
   Shape.—Ovate to lanceolate.
   Apex.—Acuminate.
   Base.—Rounded.
   Margin.—Few shallow lobes.
   Size.—Length: 6.0 cm to 10.0 cm Width: 3.0 cm to 5.0 cm.
   Texture (both surfaces).—Smooth.
   Vein color.—Upper surface: RHS 1C Lower surface: RHS 149D.
   Bract color.—Upper surface: RHS 1C, center is between RHS 51B and RHS 51C Lower surface: RHS 1C, center is RHS 51A and RHS 51B.
   Bract petiole.—Length: 1.0 cm to 2.0 cm Diameter: 0.2 cm Color: Upper surface: RHS 47B Lower surface: RHS 135D, moving to reddish.
Cyme:
   Diameter.—2.0 cm to 3.0 cm.
   Cyathia number.—8 to 15 per inflorescence.
   Cyathium.—Shape: Ovate Diameter: 0.3 cm to 0.5 cm Length: 0.4 cm to 0.7 cm Color: RHS 135C and RHS 142B.
   Peduncle.—Color: RHS 142B Length: 0.1 cm to 0.2 cm.
   Nectar cups.—Number: 1 per cyathia Diameter: 0.1 cm when closed, 0.2 cm when open Size: 0.3 to 0.5 cm Color: RHS 142C when closed, RHS 13B when open.
Reproductive organs:
   Stamens.—Quantity: 10 to 15 per cyathia Shape: Ovate.
   Filament.—Length: 0.3 cm Color: RHS 1D.
   Pollen.—Quantity: Moderate Color: RHS 9A.
   Gynoecium.—Present.
Fruit and seed set: No fruit or seed have been observed
Disease and insect/pest resistance: Good

### COMPARISON WITH COMMERCIAL VARIETY

‘NPCW10184’ differs from the commercial variety ‘Marblestar’ (U.S. Plant Pat. No. 9,685) by having a pink color of RHS 47D and dark green leaves, while ‘Marblestar’ has a pink color of RHS 51B and 51C and light green leaves.

We claim:

1. A new and distinct variety of poinsettia plant named ‘NPCW10184’ as described and shown herein.
